(19) United States
(12) Patent Application Publication
(10) Pub. No.: US 2017/0291006 A1
(43) Pub. Date: Oct. 12, 2017

(54) METHOD FOR DETERMINING GASES PROPORTIONS IN AN INHALABLE MEDICAL GASEOUS COMPOSITION AND INHALATION TEMPERATURE OF SUCH INHALABLE MEDICAL GASEOUS COMPOSITION

(71) Applicant: MONATOMICS, Paris (FR)

(72) Inventor: Helene DAVID, Saint-Laurent de l'lle d'Orleans (CA)

(73) Assignee: MONATOMICS, Paris (FR)

(21) Appl. No.: 15/482,441

(22) Filed: Apr. 7, 2017

Related U.S. Application Data
(63) Continuation of application No. PCT/FR2016/050823, filed on Apr. 8, 2016.
Application No. PCT/FR2016/050823 is a continuation of application No. PCT/FR2016/050824, filed on Apr. 8, 2016.

(51) Int. Cl. A61M 16/12; G06F 19/00; A61F 7/00

(52) U.S. Cl. CPC A61M 16/12; A61F 7/00; G06F 19/325; A61M 2202/0208; A61M 2202/0291; A61M 2202/025

(57) ABSTRACT

A method for determining gases proportions to obtain an inhalable medical gaseous composition consisting of a first noble gas, a second noble gas, and oxygen, in order to reach a body temperature value, including determining a proportion of first or second noble gas depending on predetermined parameters, such as the body temperature value and an inhalation temperature value, the determining further including determining, for the inhalation temperature value, two body temperature theoretical values for the first and second noble gas modeled by reference regression lines, calculating a difference between both body temperature theoretical values, calculating a difference between body temperature value and any one of the body temperature theoretical values, calculating a ratio representing proportion of first or second noble gas in the inhalable medical gaseous composition.

### TECHNICAL FIELD

This invention relates to a method for determining, in an inhalable medical gaseous composition containing at least two noble gases and a chosen proportion of oxygen, proportions of said two noble gases as well as the inhalation temperature of the gaseous composition to be administered to a mammal in order to reach a body temperature target value for said mammal.

### BACKGROUND ART

Among the noble gases, xenon, argon, and helium have been repeatedly shown to possess organ-protective, neuroprotective, and therapeutic properties in animal models of human diseases, such as heart attack, renal ischemia, ischemic stroke, brain trauma, and addiction.

However, from a thermal point of view, these gases differ and even oppose one another. While Xenon and Argon have both a higher molar mass and a lower thermal conductivity and specific heat than Nitrogen which is the main diluent of oxygen in air, Helium in contrast has a lower molar mass and a higher thermal conductivity and specific heat than Nitrogen. So, when these gases are administered by inhalation, for example, the body temperature can be modified according to the type of the noble gas inhaled and the temperature of inhalation of the gases. Accordingly, compared to nitrogen and depending on inhalation temperature, breathing helium reduces body temperature while xenon and argon increase it.

It could be important to reach a body temperature target value during the treatment of some diseases. For instance, clinical trials are now being performed to determine the neuroprotective effect of hypothermia in patients suffering acute ischemic stroke. Also, experimental studies in rodents have shown that combining hypothermia and xenon allows providing similar—or increasing—neuroprotection while reducing xenon concentration. Currently, in most cases, body temperature is controlled through external mechanical devices such as cooling/heating blankets. To the best of our knowledge, no one has reported a method for controlling body temperature and reaching a body temperature target value in mammals treated with organ-protective noble gases with higher molar mass and a lower thermal conductivity and specific heat than nitrogen, such as xenon and argon, by combining these gases with helium at specific concentrations and inhalation temperatures.

Accordingly, it is an object of this invention to provide a method for determining in an inhalable medical gaseous composition containing at least two noble gases A and B and a chosen volume proportion of oxygen, the volume proportions of said gases A and B in order to reach a body temperature target value, or the inhalation temperature of the medical gaseous composition to be administered to a patient in order to reach the body temperature target value in said patient.

### SUMMARY OF THE INVENTION

The present invention is directed to, in a first embodiment, a method for determining gases proportions to obtain an inhalable medical gaseous composition consisting of three gases including a first noble gas, a second noble gas and oxygen, in order to reach a body temperature target value, the method comprising:
a stage for determining one proportion of first noble gas or second noble gas in said inhalable medical gaseous composition, said stage depending on predetermined parameters including said body temperature target value and an inhalation temperature value of said inhalable medical gaseous composition, said stage comprising:
a first step for determining, for said inhalation temperature value, a first body temperature theoretical value, for the first noble gas modeled by a first reference regression line,
a second step for determining for said inhalation temperature value, a second body temperature theoretical value, for the second noble gas modeled by a second reference regression line,
a third step for calculating a highest gap as a difference between the first body temperature theoretical value and the second body temperature theoretical value,
a fourth step for calculating a target gap as a difference between said body temperature target value and any one of the first body temperature theoretical value and the second body temperature theoretical value,
a fifth step for calculating a ratio between said target gap and said highest gap, said ratio representing said proportion of first noble gas or second noble gas in said inhalable medical gaseous composition.

This first embodiment is particularly advantageous to reach directly any body temperature target value, while avoiding step by step manual process wherein medical staff has first to choose gases proportion, then to apply such a composition to a patient, then to measure the resulting body temperature value and to modify gases proportion to reach the body temperature target value. The time for doing such a step by step process may be a problem in a medical context, and the method of the invention enables to reach at the first time the body temperature target value, considering possible approximations.

In other words, the first, second and third steps can be also said as: a first step for determining the body temperature theoretical value for a given noble gas and a given inhalation temperature value of the given noble gas, as modeled by a first reference regression line, a second step for determining the body temperature theoretical value for another given noble gas and a given inhalation temperature value of said another given noble gas, as modeled by a second reference regression line, and a third step for calculating the difference gap between the first body temperature theoretical value and the second body temperature theoretical value.

It should be noted, that a gaseous composition is said inhalable, when said composition comprises a proportion of at least 21% oxygen. Generally, “proportion” can be heard by volume proportion.

According to an implementation, predetermined parameters comprise also an oxygen proportion in the inhalable medical gaseous composition, said method further comprises an additional step, a sixth step, for calculating said first noble gas or second noble gas proportion taking into account the oxygen proportion in said inhalable medical gaseous composition. More particularly, this sixth step depends on the ratio and the oxygen proportion.

According to an implementation, the sixth step for calculating said first noble gas or second noble gas proportion in the inhalable medical gaseous composition is realized by multiplication of the ratio by a proportion equal to 100% minus oxygen proportion.

According to an implementation, an additional step, a seventh step, for calculating the first noble gas or second noble gas proportion is realized by subtracting respectively the second noble gas or the first noble gas proportion to the proportion equal to 100% minus oxygen proportion.

According to an implementation, the first noble gas is selected from a first group of noble gases having a higher molar mass than molar mass of Nitrogen and a lower thermal conductivity and specific heat than thermal conductivity and specific heat of Nitrogen, and the second noble gas is selected from a second group of noble gases having a lower molar mass than molar mass of Nitrogen and a higher thermal conductivity and specific heat than thermal conductivity and specific heat of Nitrogen.

In other words, the first group is hyperthermic noble gases group and the second group is hypothermic noble gases.

According to an implementation, the first group consists of Xenon or Argon.

According to an implementation, the second group consists of Helium or Neon.

According to an implementation, the first reference regression line is a reference regression line for Xenon, said reference regression line making connection between Xenon proportions, said inhalation temperature value, and said body temperature target value.

According to an implementation, the first reference regression line is a reference regression line for Argon, said reference regression line making connection between Argon proportions, said inhalation temperature value, and said body temperature target value.

According to an implementation, the second reference regression line is a reference regression line for Helium, said reference regression line making connection between Helium proportions, said inhalation temperature value, and said body temperature target value.

According to an implementation, the oxygen proportion is at least 21% of oxygen.

According to an implementation, the body temperature target value is comprised between 32° Celsius and 38° Celsius.

According to an implementation, a measurement uncertainty of plus/minus X° Celsius on the inhalation temperature value corresponding to a measurement uncertainty of plus/minus X° Celsius/2 on the body temperature target value.

The present invention is also directed to, in a second embodiment, a method for determining an inhalation temperature value for an inhalable medical gaseous composition, consisting of three gases including a first noble gas, a second noble gas and oxygen, in order to reach a body temperature target value depending on parameters including proportion of first noble gas, proportion of second noble gas, and said body temperature target value, wherein said method comprises a setting up of a proportion table wherein gases proportions are indicated for several body temperature target values and inhalation temperature values, at least one gas proportion being calculated by steps comprising:
a first step for determining, for a given inhalation temperature value, a first body temperature theoretical value, for the first noble gas modeled by a first reference regression line,
a second step for determining for said given inhalation temperature value, a second body temperature theoretical value, for the second noble gas modeled by a second reference regression line,
a third step for calculating a highest gap as a difference between the first body temperature theoretical value and the second body temperature theoretical value,
a fourth step for calculating a target gap as a difference between said body temperature target value and any one of the first body temperature theoretical value and the second body temperature theoretical value,
a fifth step for calculating a ratio between said target gap and said highest gap, said ratio representing said proportion of first noble gas or second noble gas in said inhalable medical gaseous composition, and
a sixth step for calculating said first noble gas or second noble gas proportion taking into account the oxygen proportion in said inhalable medical gaseous composition.

This second embodiment is particularly advantageous to indicate to which temperature the inhalable medical gaseous composition has to be inhaled in order to reach the body temperature target value.

According to an implementation, the sixth step for calculating said first noble gas or second noble gas proportion in the inhalable medical gaseous composition is calculated by multiplication of the ratio obtained for first noble gas or second noble gas by a volume proportion equal to 100% minus oxygen proportion.

According to an implementation, the first noble gas is selected from a first group of noble gases having a higher molar mass than molar mass of Nitrogen and a lower thermal conductivity and specific heat than thermal conductivity and specific heat of Nitrogen, and the second noble gas is selected from a second group of noble gases having a lower molar mass than molar mass of Nitrogen and a higher thermal conductivity and specific heat than thermal conductivity and specific heat of Nitrogen.

In other words, the first group is hyperthermic noble gases group and the second group is hypothermic noble gases.

According to an implementation, the first group consists of Xenon or Argon.

According to an implementation, the second group consists of Helium or Neon.

According to an implementation, a measurement uncertainty of plus/minus X° Celsius on the body temperature target value corresponding to a measurement uncertainty of plus/minus 2X° Celsius on the inhalation temperature value.

The present invention is also directed to, in another embodiment, a computing device configured to implement a method for determining gases proportions to obtain an inhalable medical gaseous composition consisting of three gases including a first noble gas, a second noble gas and oxygen, in order to reach a body temperature target value:
wherein computing device is suitable for determining a proportion of first noble gas or second noble gas of said inhalable medical gaseous composition, depending on predetermined parameters including said body temperature target value, an inhalation temperature value of said inhalable medical gaseous composition and an oxygen proportion in the inhalable medical gaseous composition, said determining proportion of first noble gas or second noble gas comprising:
a first step for determining, for said inhalation temperature value, a first body temperature theoretical value, for the first noble gas modeled by a first reference regression line,
a second step for determining for said inhalation temperature value, a second body temperature theoretical value, for the second noble gas modeled by a second reference regression line,
a third step for calculating a highest gap as a difference between the first body temperature theoretical value and the second body temperature theoretical value,
a fourth step for calculating a target gap as a difference between said body temperature target value and any one of the first body temperature theoretical value and the second body temperature theoretical value,
a fifth step for calculating a ratio between said target gap and said highest gap, said ratio representing said proportion of first noble gas or second noble gas in said inhalable medical gaseous composition, and
a sixth step for calculating said first noble gas or second noble gas proportion taking into account the oxygen proportion in said inhalable medical gaseous composition.

According to an implementation, the sixth step for calculating said first noble gas or second noble gas proportion in the inhalable medical gaseous composition is calculated by multiplication of the ratio obtained for first noble gas or second noble gas by a volume proportion equal to 100% minus oxygen proportion.

According to an implementation, said computing device is also configured to implement a method for determining an inhalation temperature value for an inhalable medical gaseous composition consisting of three gases including a first noble gas, a second noble gas and oxygen, in order to reach a body temperature target value depending on parameters including proportions of first noble gas and a second noble gas, and said body temperature target value, wherein computing device is suitable for setting up of a proportion table.

According to an implementation, computing device is configured with both first and second reference regression lines.

According to an implementation, the sixth step for calculating said first noble gas or second noble gas proportion in the inhalable medical gaseous composition is calculated by multiplication of the ratio obtained for first noble gas or second noble gas by a volume proportion equal to 100% minus oxygen proportion.

### BRIEF DESCRIPTION OF THE DRAWINGS

For the present invention to be clearly understood and readily practiced, the present invention will be described in conjunction with the following figures, wherein:

FIG. 1 is a table showing the physical properties of the compounds of the present invention,

FIG. 2 is a graphical representation of a rat body temperature depending on an inhalation temperature of the inhaled gas, which is helium (curve C1) or xenon (curve C2);

FIG. 3 is a graphical representation of a rat body temperature depending on an inhalation temperature of the inhaled gas, which is helium (curve C1) or argon (curve C3);

FIG. 4 is a flow diagram illustrating a method for determining gases proportions to obtain an inhalable medical gaseous composition according to the first embodiment of the present invention,

FIG. 5 is a table showing proportions of xenon and helium depending on predetermined parameters, like oxygen proportion, inhalation temperature of the gaseous composition and body temperature target value, this table being taken from graphical representation of FIG. 2.

FIG. 6 which has been obtained from the same experimental value than FIG. 2, shows the graphical representation of the part of Helium at Tii=22° C., which part of Helium is shifted to the right or to the left to determine the range in-between Tii should be maintained to control the body temperature target value BT within an incertitude of 0.5° C. and 1° C.

### DETAILED DESCRIPTION OF THE INVENTION

It is to be understood that the figures and description of the present invention illustrate elements that are relevant for a clear understanding of the present invention, while eliminating, for purposes of clarity, other elements. Those of ordinary skill in the art will recognize that other elements may be desirable to produce an operational system incorporating the present invention. However, because such elements are well known in the art, and because they do not facilitate a better understanding of the present invention, a discussion of such elements is not provided herein.

First of all, it should be noted that noble gases such as Xenon, Argon, and Helium have been repeatedly shown to possess organ-protective, neuroprotective, and therapeutic properties in animal models of human diseases, such as heart attack, renal ischemia, ischemic stroke, brain trauma, and addiction. Moreover, noble gases have the advantage to be not metabolized after to be inhaled. According to a first embodiment, the invention is about a method for determining proportions of these noble gases in an inhalable medical gaseous composition.

The inhalable medical gaseous composition consists of three gases including a first noble gas, a second noble gas and oxygen. To be inhalable this composition includes at least 21% oxygen in volume proportion, which is the volume proportion of oxygen in air, so that there is at the most 79% of noble gases in volume proportion in the inhalable gaseous composition, the noble gas mixture replacing nitrogen, which concentration in air can be considered equal to 79% in volume proportion.

The noble gas mixture consists of a first noble gas and a second noble gas. The first noble gas is selected from a first group of noble gases whose molar mass, and thermal conductivity and specific heat, are respectively higher and lower than those of air and the second noble gas is selected from a second group of noble gases which molar mass, and thermal conductivity and specific heat, are respectively lower and higher that those of air. Proportions of each noble gas are such that the resulting molar mass, thermal conductivity and specific heat, of the inhalable medical gaseous composition enables to reach, depending on inhalation temperature, a body temperature target value. For example, this body temperature target value is comprised between 32° Celsius and 38° Celsius.

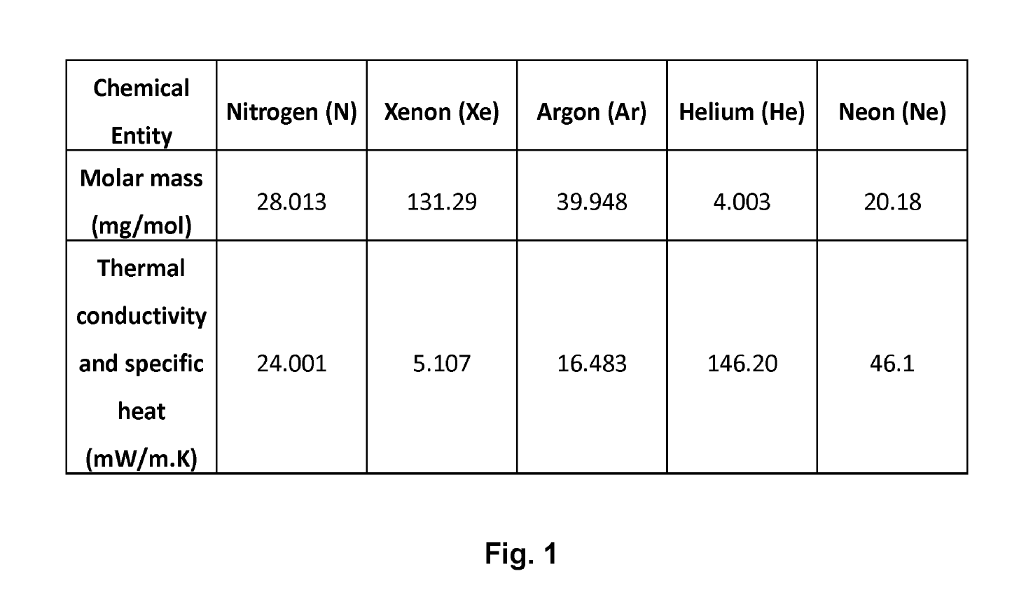

Noble gases with a higher molar mass and a lower thermal conductivity and specific heat than Nitrogen, the main diluent of oxygen in air, include Xenon or Argon (FIG. 1).

Noble gases with a lower molar mass and a higher thermal conductivity and specific heat than Nitrogen include Helium and Neon (FIG. 1). Advantageously, hypothermia induced by a Helium-containing gas mixture, supposed to diffuse homogeneously within the body, would be likely to avoid or at least to reduce gradient temperature within the body compared to mechanical techniques.

To reach the body temperature target value, proportions of each noble gases in the noble gases mixture, or in the inhalable medical gaseous composition, have to be precisely calculated. More particularly, proportions of each noble gas are such that the resulting molar mass, thermal conductivity and specific heat of the inhalable medical gaseous composition enables to reach, depending on inhalation temperature, a body temperature target value. These proportions are extrapolated from experimental data realized with precited noble gases.

Experimental data were obtained as follows: Male adult Sprague-Dawley rats, weighing 250-280 g, were used. Rats were placed in a closed chamber of 10 L volume, fitted with a viewing window. The effects on the rats' body temperature of a 3-h treatment with Xenon-Oxygen, Argon-Oxygen, or Helium-Oxygen gas mixtures containing 22% Oxygen in volume proportion at various gas inhalation temperatures were investigated (3-4 per group). The gas flow rate was 6 L/min, a condition that allows maintaining carbon dioxide at a concentration below 0.03%. The temperature at which the gas mixtures were administered was controlled using a gas heating/cooling system and a temperature probe placed in the closed chamber. Immediately before and after the 3-h period of exposure to the above-mentioned gas mixtures, the rats' body temperature was measured.

Forty rats were obtained from Janvier Lab (Le Genest Saint-Isle, France). All animal use procedures were approved by the local ethic committee in accordance with the framework of the French legislation for biomedical experimentation and The European Communities Council Directive issued on 24 Nov. 1986 (86/609/EEC). Before being used, rats were housed at 21±0.5° C. in Perspex home cages with free access to food and water. Light was maintained on a light/dark reverse cycle with lights on from 8:00 p.m. to 8:00 a.m.)

Xenon, Argon, Helium, and Oxygen of medicinal grade were obtained from Air Liquide Santé (Paris, France). Gas mixtures containing Xenon, Argon, Helium, or Nitrogen, in combination with 22% Oxygen, were obtained using computer-driven gas mass flowmeters (Aalborg, Orangeburg, N.Y., USA) of 1% absolute accuracy and an oxygen analyzer.

It should be noted that rodents, such as rats are usually used as models in preclinical studies investigating the mechanisms and possible treatments of human pathologies. Moreover, rats' average body temperature and humans' average body temperature are in the same range, so that administration of inhalable medical gaseous composition to rats may be likened to an administration of inhalable medical gaseous composition to humans.

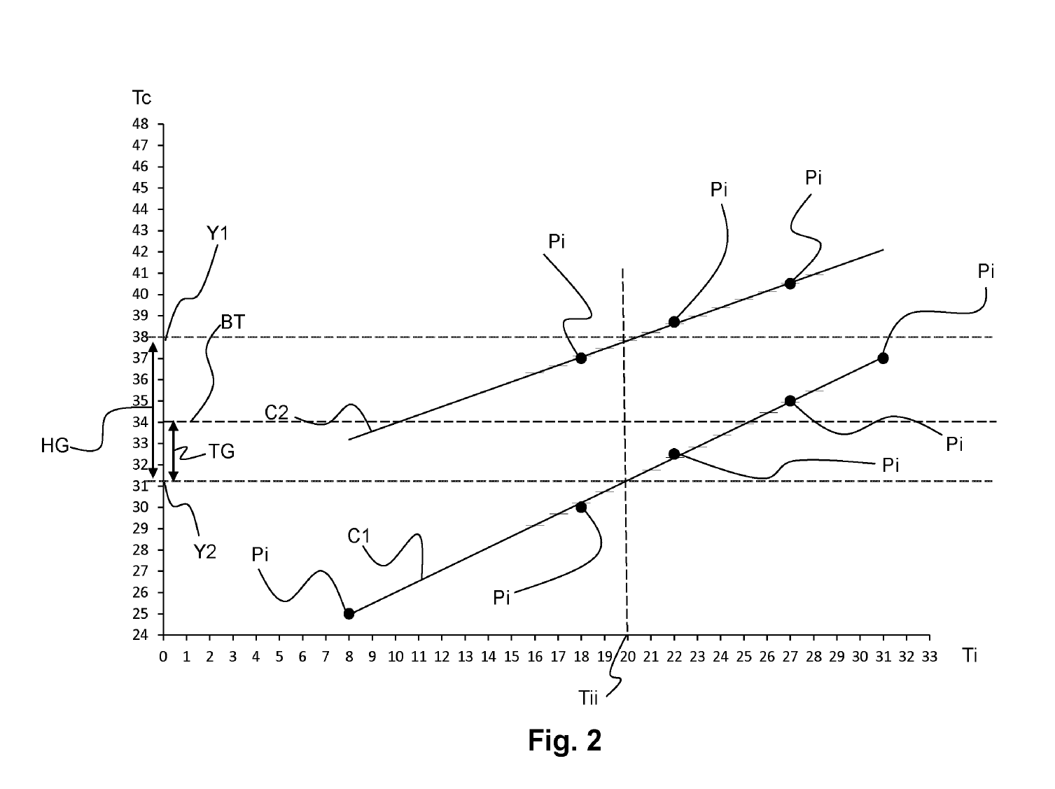

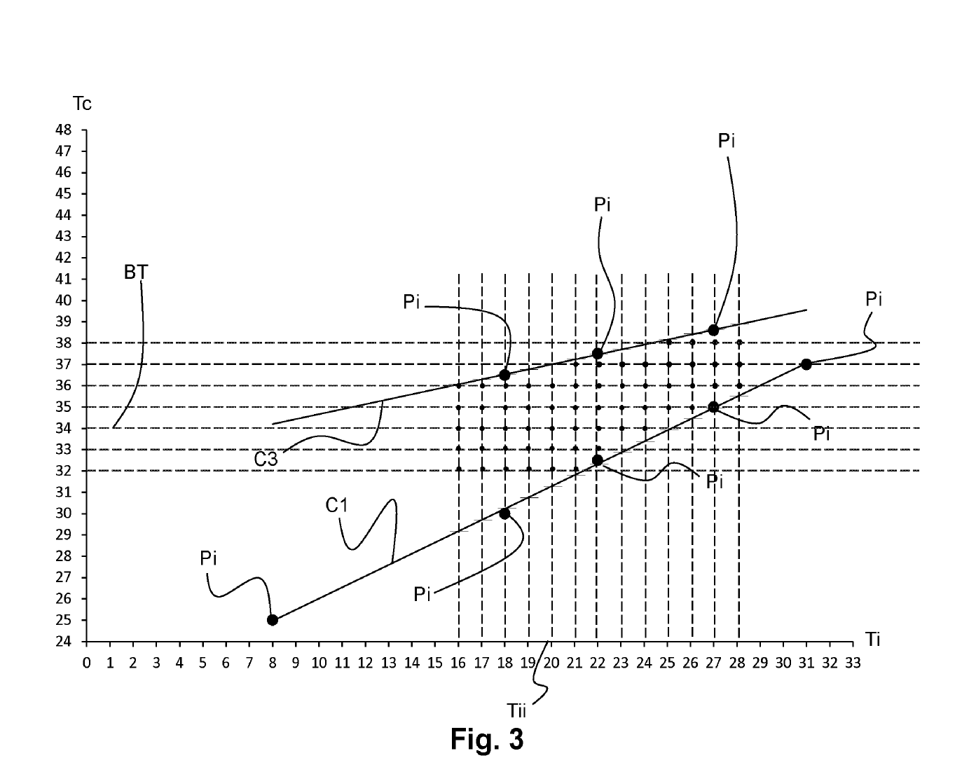

Graphic representations of FIG. 2 and FIG. 3 have been obtained from these experimental data and consist in regression lines of experimental point Pi. More precisely, FIG. 2 shows the rats' body temperature Tc as a function gas mixture inhalation temperature Ti for a gas mixture including 78% Helium-22% Oxygen (curve C1) and for a gas mixture including 78% Xenon-22% Oxygen (curve C2). FIG. 3 shows the rats' body temperature Tc as a function of the gas mixture inhalation temperature Ti for a gas mixture including 78% Helium-22% Oxygen (curve C1) and for a gas mixture including 78% Argon-22% Oxygen (curve C3). Curves C1, C2, C3 are regression lines obtained from experimental data Pi and are used as reference regression lines. It should be noted that reference regression line Helium-Oxygen C1 is the same on FIGS. 2 and 3.

It should be noted that the reference regression line C1 for Helium makes connection between Helium proportions, inhalation temperature Ti, and body temperature Tc. In the same manner, regression line C2 for Xenon makes connection between Xenon proportions, inhalation temperature Ti, and body temperature Tc, and regression line for Argon C3 makes connection between Argon proportions, inhalation temperature Ti, and body temperature Tc.

According to FIGS. 2 and 3, reference regression line Helium-Oxygen C1 has an affine function y=0.526x+20.748, reference regression line Xenon-Oxygen C2 has an affine function y=0.3877x+30.075 and reference regression line Argon-Oxygen C3 has an affine function y=0.2328x+32.334. Of course, these affine functions are given for example.

Hereinafter, an example of the method for determining proportions of gases, according to the first embodiment, is illustrated by calculating proportions of gases in a noble gas mixture containing Xenon and Helium that will allow reaching a body temperature target value BT while breathing the noble gas mixture at a given temperature of gas inhalation Ti. Of course, this example also works with a Helium-Argon mixture or any other kind of hyperthermic noble gas and hypothermic noble gas mixture.

In order to illustrate the method according to an aspect of the invention we provide example of determination of the proportions of gases depending on predetermined parameters such as an inhalation temperature Tii at which the gas mixture is administered and the body temperature target value BT. The body temperature target value is drawn as a horizontal line at 34° C. on FIGS. 2 and 3, and labelled BT. For the example, the body temperature value target BT is equal to 34° C. and the inhalation temperature value Tii is equal to 20° C.

The method comprises:

A first step S1 for determining, for said inhalation temperature value Tii, a first body temperature theoretical value Y1, using reference regression line C2 for Xenon-Oxygen mixture for example. According to this example, the first body temperature theoretical value Y1 is:

Y1=0.3877×20+30.075=37.829° C.

A second step S2 for determining, for said inhalation temperature value Tii, a second body temperature theoretical value Y2, using reference regression line C1 for Helium-Oxygen mixture. According to the example, the second body temperature theoretical value Y2 is:

Y2=0.526×20+20.748=31.268° C.

A third step S3 for calculating a highest gap HG as a difference between the first body temperature theoretical value Y1 and the second body temperature theoretical value Y2. According to the example, highest gap HG is:

HG=Y1−Y2=37.829−31.268=6.561° C.

A fourth step S4 for calculating a target gap TG as a difference between said body temperature target value BT and any one of the first body temperature theoretical value Y1 and the second body temperature theoretical value Y2. According to the example, the second body temperature theoretical value Y2 is arbitrarily taken, the body temperature target value BT is 34° C., and the target gap TG is:

TG=BT−Y2=34−31.268=2.732° C.

It has to be noted that, if the first or second body temperature theoretical value is bigger than the body temperature target value BT, the absolute value is taken for calculating he target gap TG.

A fifth step S5 for calculating a ratio R between the previously determined target gap TG and the previously determined highest gap HG. The ratio R represents the proportion of Xenon, or Argon, and Helium in the noble gases mixture according to the choice of the first body temperature theoretical value Y1 or the second body temperature theoretical value Y2 in the forward step. According to the example, the ratio R is:

R=TG/HG=2.732/6.561=41.64% of Xenon.

It results from such a method that the noble gas mixture here comprises 41.64% of Xenon and then 58.36% of Helium.

In order to be inhaled and to obtain an inhalable medical gaseous composition, the noble gas mixture has to be mixed with oxygen. In the inhalable medical gaseous composition, proportion of oxygen O2 is at least 21% and at the most 50%. Preferably, proportion of oxygen O2 in the inhalable medical gaseous composition is between 21% and 30% or between 21% and 25%.

According to a variant of the first embodiment of the invention, the method further comprises a sixth step S6 for calculating proportion of one, for example Xenon, among the three gases in the inhalable medical gaseous composition. In such a sixth step, the ratio R is multiplied by a proportion equal to 100% minus the proportion of oxygen O2.

For instance, if volume proportion of oxygen O2 is 22%, according to the previous example, the ratio R is equal to:

R×(100%−O2)=41.64%×78%=32.48%.

According to this example the inhalable medical gaseous composition comprises 32.48% Xenon, 22% Oxygen and 45.52% Helium. To obtain a body temperature target value BT of 34° C., a gas mixture containing 22% Oxygen should be composed of 46% Helium and 32% Xenon if inhalation temperature value Tii is 20° C. In other words, inhalation of this composition, with an inhalation temperature value Tii equal to 20° C., allows reaching the body temperature target value BT of 34° C.

The further described embodiment of the method of the invention enables to determine gases proportion on the basis of parameters such as a body temperature target value, an inhalation temperature value and eventually a fixed proportion of oxygen. It has to be noted that some approximations may be done about gas proportions considering that reference regression lines C1, C2, C3 used for the method are regression lines.

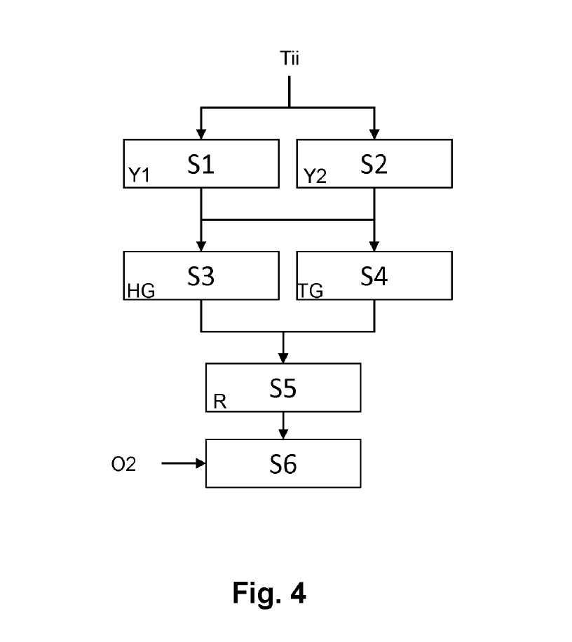

FIG. 4 illustrates the flow chart of the method for determining volume proportions of gases according to the first embodiment. Steps S1 and S2 for determining body temperature theoretical values Y1, Y2, as represented, can be realized simultaneously. Then, third step S3 for calculating the highest gap HG and fourth step S4 for calculating a target gap TG, as represented, can be realized simultaneously. These steps S3 and S4 are necessarily realized after steps S1 and S2. Fifth step S5 for calculating ratio R between the target gap TG and the highest gap HG is necessarily realized after third step S3. Finally, to obtain gas proportions in the inhalable medical gaseous composition, method comprises the sixth step S6 for calculating proportion of one among the three gases in the inhalable medical gaseous composition necessarily realized after step 5.

The same method is applied from FIG. 3 for determining the volume proportions of gases in the noble gas mixture containing Argon and Helium or in an inhalable medical gaseous composition containing Argon, Helium and Oxygen with reference regression line C3 of Argon-Oxygen.

According to a second embodiment, the invention is about a method for determining the inhalation temperature value Tii of the inhalable medical gaseous composition whose gas proportions are known to reach a given body temperature target value BT. This second embodiment is particularly advantageous to determine at which temperature the inhalable medical gaseous composition has to be inhaled in order to reach the body temperature target value BT.

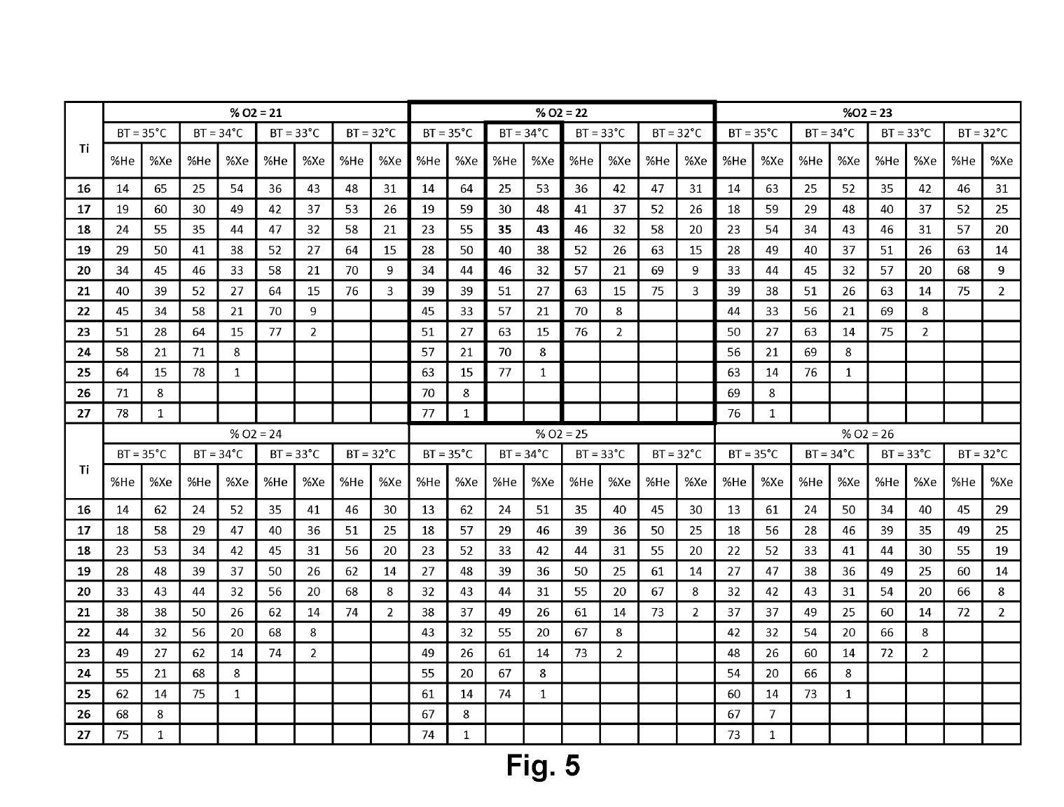

The method of determining the inhalation temperature value Tii of the inhalable medical gaseous composition comprises a setting up of a proportion table, representing proportions of noble gases depending on the volume proportion of oxygen O2, the body target temperature BT and inhalation temperature Tii. An example of this kind of proportion table is show in FIG. 5 for an inhalable medical gaseous composition consisting of Argon-Helium-Oxygen. It has to be noted that the proportion table is realized according to the method used for determining volume proportions of gases according to the first embodiment, each gas proportion value being calculated with at least the five steps S1 to S5 hereinabove described.

Such a proportion table allows determining the inhalation temperature value Tii for a given inhalable medical gaseous composition. More precisely, this proportion table can be used in various ways. According to a first way, the proportion table is compared with volume proportions of the inhalable medical gaseous composition by a clinician who is in charge to administrate the inhalable medical gaseous composition in order to reach a body temperature target value BT. For example, for a gaseous composition comprising 43% Xe-35% He-22% O2 in volume proportion and the body temperature target value BT is about 34° C., the clinician reads the proportion table and seeks 43% Xe-35% He-22% O2 in column of BT=34° C. The clinician reads that the inhalation temperature value Tii is 18° C. So, the clinician will take care administering the inhalable medical gaseous composition to 18° C. in order to obtain a 34° C. the body temperature target value BT.

According to a second way, the proportion table is compared with the volume proportions of the inhalable medical gaseous composition and with body temperature target value BT by a computing device. The computing device is configured to display the inhalation temperature value Tii in order to reach the body temperature target value BT.

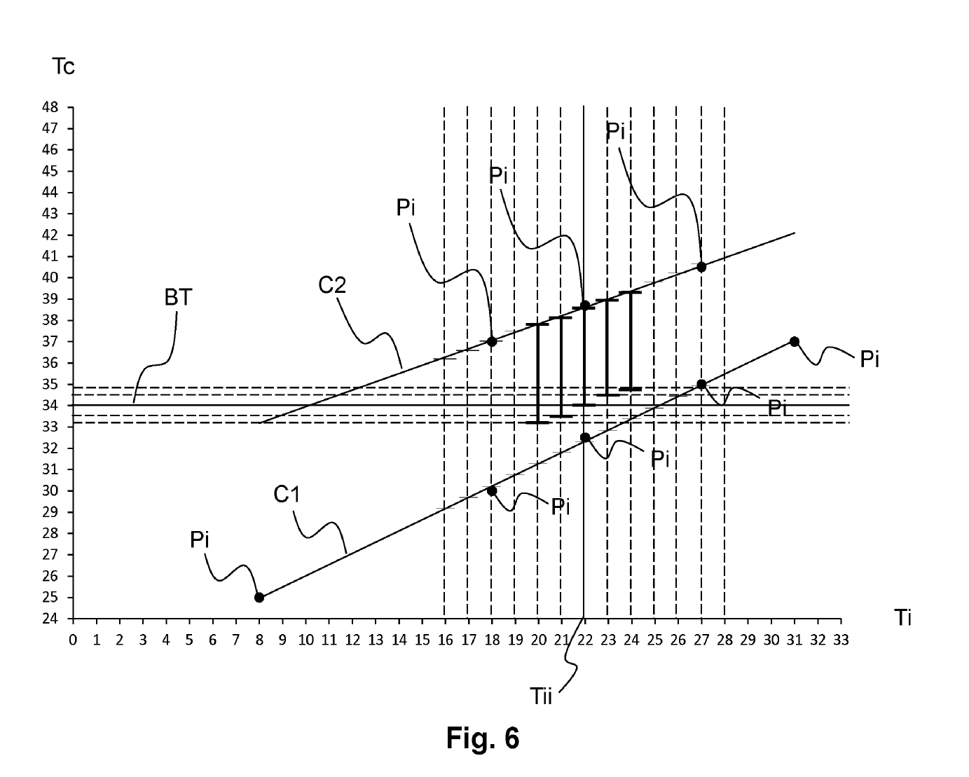

It should be noted, in relation with FIG. 6, that a given measurement uncertainty on the body temperature target value BT represents another measurement uncertainty on the inhalation temperature value Tii and conversely. More precisely, a 0.5° C. measurement uncertainty on the body temperature target value BT represents 1° C. measurement uncertainty on the inhalation temperature Tii. Indeed, on FIG. 6 it is noticeable that for a same proportion of noble gases and for a 33.5° C. body temperature target value BT, the inhalation temperature value Tii is 21° C., whereas for a 34.5° C. body temperature target value BT, the inhalation temperature value Tii is 23° C. In the same manner, a 1° C. measurement uncertainty on the body temperature target value BT represents 2° C. measurement uncertainty on the inhalation temperature Tii. The measurement uncertainty is quite linear, so that a 2° C. measurement uncertainty on the body temperature target value BT represents approximately 4° C. measurement uncertainty on the inhalation temperature Tii. So, to generalize a measurement uncertainty of plus/minus X degree Celsius on the body temperature target value BT corresponds to a measurement uncertainty of plus/minus 2 multiplied by X° Celsius on the inhalation temperature value Tii.

Accordingly, the example above, to obtain a body temperature target value BT of 34° C. plus/minus 0.5° C., a clinician will have to maintain the gas inhalation temperature Tii between 21° C. and 23° C. In the same manner, to obtain a body temperature target value BT of 34° C. plus/minus 1° C., a clinician will have to maintain the gas inhalation temperature Tii between 20° C. and 24° C.

Methods described hereabove can be implemented by a computing device including specifically programmed processing circuitry.

To run the method for determining proportions of gases on the basis of a known inhalation temperature value, the computing device is suitable for receiving predetermined parameters such as said inhalation temperature value Tii, body temperature target value BT, and potentially the volume proportion of oxygen O2.

In the same manner, the method for determining the inhalation temperature Tii of the inhalable medical gaseous composition can be also implemented by a computing device which is suitable for receiving predetermined parameters such as the volume proportions of gases in a noble gas mixture or in an inhalable medical gaseous composition and the body temperature target value BT.

1. A method for determining gases proportions to obtain an inhalable medical gaseous composition consisting of three gases including a first noble gas, a second noble gas, and oxygen, in order to reach a body temperature target value, the method comprising:
determining one proportion of first noble gas or second noble gas in said inhalable medical gaseous composition, said determining depending on predetermined parameters including said body temperature target value and an inhalation temperature value of said inhalable medical gaseous composition, said determining further comprising:
determining, for said inhalation temperature value, a first body temperature theoretical value, for the first noble gas modeled by a first reference regression line,
determining. for said inhalation temperature value, a second body temperature theoretical value, for the second noble gas modeled by a second reference regression line,
calculating a highest gap as a difference between the first body temperature theoretical value and the second body temperature theoretical value,
calculating a target gap as a difference between said body temperature target value and any one of the first body temperature theoretical value and the second body temperature theoretical value, and
calculating a ratio between said target gap and said highest gap, said ratio representing said proportion of first noble gas or second noble gas in said inhalable medical gaseous composition.

2. The method according to claim 1, further comprising:
administering the inhalable medical gaseous composition with the proportion of the first noble gas or the second noble gas to a patient in order to reach the body temperature target value in said patient.

3. The method according to claim 1, wherein the predetermined parameters further comprise an oxygen proportion in the inhalable medical gaseous composition, and wherein said method further comprises calculating said first noble gas or second noble gas proportion taking into account the oxygen proportion in said inhalable medical gaseous composition.

4. The method according to claim 3, wherein the calculating of said first noble gas or second noble gas proportion in the inhalable medical gaseous composition is realized by multiplication of the ratio by a proportion equal to 100% minus oxygen proportion.

5. The method according to claim 4, further comprising calculating the first noble gas or second noble gas proportion by subtracting respectively the second noble gas or the first noble gas proportion to the proportion equal to 100% minus oxygen proportion.

6. The method according to claim 1, wherein the first noble gas is selected from a first group of noble gases having a higher molar mass than molar mass of Nitrogen and a lower thermal conductivity and specific heat than thermal conductivity and specific heat of Nitrogen, and the second noble gas is selected from a second group of noble gases having a lower molar mass than molar mass of Nitrogen and a higher thermal conductivity and specific heat than thermal conductivity and specific heat of Nitrogen.

7. The method according to claim 6, wherein the first group consists of Xenon or Argon.

8. The method according to claim 6, wherein the second group consists of Helium or Neon.

9. The method according to claim 1, wherein the first reference regression line is a reference regression line for Xenon, said reference regression line making connection between Xenon proportions, said inhalation temperature value, and said body temperature target value.

10. The method according to claim 1, wherein the first reference regression line is a reference regression line for Argon, said reference regression line making connection between Argon proportions, said inhalation temperature value, and said body temperature target value.

11. The method according to claim 8 wherein the second reference regression line is a reference regression line for Helium, said reference regression line making connection between Helium proportions, said inhalation temperature value, and said body temperature target value.

12. The method according to claim 1, wherein the oxygen proportion is at least 21% of oxygen.

13. The method according to claim 1, wherein the body temperature target value is comprised between 32° Celsius and 38° Celsius.

14. The method according to claim 1, wherein a measurement uncertainty of plus/minus X° Celsius on the inhalation temperature value corresponding to a measurement uncertainty of plus/minus
$$\frac{X ° Celsius}{2}$$
on the body temperature target value.

15. A method for determining an inhalation temperature value for an inhalable medical gaseous composition, consisting of three gases including a first noble gas, a second noble gas and oxygen, in order to reach a body temperature target value depending on parameters including proportion of first noble gas, proportion of second noble gas, oxygen proportion and said body temperature target value, comprising:
setting up of a proportion table wherein gases proportions are indicated for several body temperature target values and inhalation temperature values, at least one gas proportion being calculated by:
determining, for a given inhalation temperature value of the inhalable medical gaseous composition, a first body temperature theoretical value, for the first noble gas modeled by a first reference regression line,
determining for said given inhalation temperature value, a second body temperature theoretical value, for the second noble gas modeled by a second reference regression line,
calculating a highest gap as a difference between the first body temperature theoretical value and the second body temperature theoretical value,
calculating a target gap as a difference between said body temperature target value and any one of the first body temperature theoretical value and the second body temperature theoretical value,
calculating a ratio between said target gap and said highest gap, said ratio representing said proportion of first noble gas or second noble gas in said inhalable medical gaseous composition, and
calculating said first noble gas or second noble gas proportion taking into account the oxygen proportion in said inhalable medical gaseous composition.

16. The method according to claim 1, further comprising:
administering the inhalable medical gaseous composition with the proportion of the first noble gas or the second noble gas to a patient in order to reach the body temperature target value in said patient.

17. The method according to claim 15, wherein the calculating said first noble gas or second noble gas proportion in the inhalable medical gaseous composition is calculated by multiplication of the ratio obtained for first noble gas or second noble gas by a volume proportion equal to 100% minus oxygen proportion.

18. The method according to claim 15, wherein the first noble gas is selected from a first group of noble gases having a higher molar mass than molar mass of Nitrogen and a lower thermal conductivity and specific heat than thermal conductivity and specific heat of Nitrogen, and the second noble gas is selected from a second group of noble gases having a lower molar mass than molar mass of Nitrogen and a higher thermal conductivity and specific heat than thermal conductivity and specific heat of Nitrogen.

19. The method according to claim 17, wherein the first group consists of Xenon or Argon.

20. The method according to claim 17, wherein the second group consists of Helium or Neon

21. The method according to claim 15, wherein a measurement uncertainty of plus/minus X° Celsius on the body temperature target value corresponding to a measurement uncertainty of plus/minus 2X° Celsius on the inhalation temperature value.

22. A computing device, comprising:
processing circuitry configured to
determine gases proportions to obtain an inhalable medical gaseous composition consisting of three gases including a first noble gas, a second noble gas and oxygen, in order to reach a body temperature target value,
wherein the processing circuitry is further configured to determine a proportion of a first noble gas or a second noble gas of said inhalable medical gaseous composition, depending on predetermined parameters including said body temperature target value, an inhalation temperature value of said inhalable medical gaseous composition, and an oxygen proportion in the inhalable medical gaseous composition,
wherein the processing circuitry is further configured to determine of the proportion of the first noble gas or the second noble gas by being further configured to
determine, for said inhalation temperature value, a first body temperature theoretical value, for the first noble gas modeled by a first reference regression line,
determine for said inhalation temperature value, a second body temperature theoretical value, for the second noble gas modeled by a second reference regression line,
calculate a highest gap as a difference between the first body temperature theoretical value and the second body temperature theoretical value,
calculate a target gap as a difference between said body temperature target value and any one of the first body temperature theoretical value and the second body temperature theoretical value,
calculate a ratio between said target gap and said highest gap, said ratio representing said proportion of first noble gas or second noble gas in said inhalable medical gaseous composition, and
calculate said first noble gas or second noble gas proportion depending on the ratio and the oxygen proportion.

23. The computing device according to claim 21, wherein the processing circuitry is further configured to calculate said first noble gas or second noble gas proportion in the inhalable medical gaseous composition by being configured to calculate multiplication of the ratio obtained for first noble gas or second noble gas by a volume proportion equal to 100% minus oxygen proportion.

24. The computing device according to claim 21, wherein said processing circuitry is further configured to determine the inhalation temperature value for the inhalable medical gaseous composition consisting of three gases including the first noble gas, the second noble gas, and oxygen, in order to reach a body temperature target value depending on parameters including proportions of the first noble gas and the second noble gas, and said body temperature target value, wherein the processing circuitry is further configured to set up a proportion table.

25. The computing device according to claim 21, wherein the processing circuitry is further configured with both first and second reference regression lines.
